(19) United States
(12) Patent Application Publication
(10) Pub. No.: US 2023/0154592 A1
(43) Pub. Date: May 18, 2023

(54) RADIOLOGY PEER REVIEW USING ARTIFICIAL INTELLIGENCE

(71) Applicant: MERATIVE US L.P., Ann Arbor, MI (US)

(72) Inventors: Dustin Michael Sargent, San Diego, CA (US); Michael Trambert, Santa Barbara, CA (US); Lenward E. Holness, JR., San Diego, CA (US); Dale Seegmiller Maudlin, Solana Beach, CA (US); Sun Young Park, San Diego, CA (US)

(21) Appl. No.: 17/530,434

(22) Filed: Nov. 18, 2021

(51) Int. Cl. G16H 30/20; G16H 50/20; G16H 40/20

(52) U.S. Cl. CPC G16H 30/20; G16H 50/20; G16H 40/20

(57) ABSTRACT

A method and system is provided for optimizing radiology peer review exam selection using artificial intelligence. The system includes an electronic processor configured to: receive a set of candidate medical imaging exams with reading physician data, assign the medical imaging exams to at least one peer reviewer, receive peer reviewer data including scores and/or text for the assigned medical imaging exams, update a machine learning algorithm to optimize the assignment of medical imaging exams to the at least one peer reviewer using the received peer reviewer data.

### FIELD

Embodiments described herein relate to systems and methods for performing peer review of medical exams using artificial intelligence.

### SUMMARY

Radiologists are required to perform peer reviews of medical exams to receive accreditation from the American College of Radiology (ACR). Peer review involves reviewers (also referred to as “colleagues” herein) reviewing images and associated reports from exams completed by a reading physician. The reviewers indicate agreement or disagreement with the findings of the reading physician. The ACR has a computer portal provided with a ratings system (RADPEER) that reviewers can use to perform peer reviews. The ACR, however, does not specify how many peer reviews must be done nor does the ACR prohibit self-review.

In addition, in practice, peer review implementation is suboptimal in terms of efficiency and quality assurance benefit. For example, many times, inefficient manual data entry is required by reviewers and a reading physician being reviewed. Also, too many reviews result in inefficient use of reviewers' time. Peer reviews are often assigned randomly, without taking consideration of the reviewers' expertise. Reviewers may also fail to review difficult exams (which may be more prone to errors) as these reviews often involve more effort and time and existing computer systems, such those provided by the ACR, do not track, monitor, or incentivize efficient and balanced reviews for complex and less complex studies, across a pool of radiologists and associated images and reports needing review.

In existing systems there is also no opportunity for a reading physician to learn from mistakes. Further, no positive feedback mechanism is provided as these systems only allow for concurring or negative comments. Aversion to criticism further inhibits review processes. In particular, RADPEER scores generated by a reviewer include: 1) concur with interpretation; 2) discrepancy in interpretation/not ordinarily expected to be made; and 3) discrepancy in interpretation/should be made most of the time. Choices 2 and 3 are either a) unlikely to be clinically significant or b) likely to be clinically significant.

Embodiments described herein address the above issues as well as other issues via an artificial intelligence (“AI”) driven picture archiving and communication system (PACS). Embodiments described herein improve efficiency and accuracy of peer review of medical imaging exams. In particular, one or more machine learning algorithms can be used to select an imaging exam for review, select a reviewer for the selected imaging exam, or both. In particular, the one or more machine learning algorithms can learn (through training data including imaging exams and associated reviews) (1) which types of exams are more prone to errors, (2) what types of mistakes each reader makes, in what situations, (3) which reviewers are good at catching mistakes on each exam type, (4) optimal peer review frequency (e.g., a balance between time spent and quality assurance benefit), or a combination thereof. Further, in some embodiments, feedback can be provided to benefit a reading physician. For example, some embodiments described herein automate positive and negative feedback to a reading physician and may also be configured to identify error trends and recommended or provided training responsive to such trends. The training may be tailored for a particular reading physician, a group of reading physicians, or the like.

For example, one embodiment provides a computer-implemented method for optimizing radiology peer review exam selection using artificial intelligence. The method includes: receiving a set of candidate medical imaging exams with reading physician data; selecting the candidate medical imaging exams for peer review; assigning the selected medical imaging exams to at least one peer reviewer; receiving peer review data from the peer reviewers assigned to the selected medical imaging exams, the peer review data including at least one score for the assigned medical imaging exams; and updating a machine learning algorithm to optimize the selection and assignment of medical imaging exams to at least one peer reviewer using the received peer review data.

Another embodiment provides a computer system for optimizing radiology peer review exam selection using artificial intelligence. The computer system includes an electronic processor; and one or more computer-readable memories. The electronic processor, through execution of instructions stored in the one or more computer-readable memories, is configured to: receive a set of candidate medical imaging exams with reading physician; select the candidate medical imaging exams for peer review; assign the selected medical imaging exams to at least one peer reviewer; receive peer review data from the at least one peer reviewer assigned to the selected medical imaging exams, the peer review data including at least one score for the assigned medical imaging exams; and update a machine learning algorithm to optimize the selection and assignment of medical imaging exams to peer reviewers using the received peer review score.

Another embodiment provides a computer program product, the computer program product comprising a non-transitory computer readable storage medium having program code. The program code is executable by an electronic processor to: receive a set of candidate medical imaging exams with reading physician data; select the candidate medical imaging exams for peer review; assign the selected medical imaging exams to peer reviewers; receive peer review data from the peer reviewers assigned to the selected medical imaging exams, the peer review data including at least one of peer review score for the assigned medical imaging exams; and update a machine learning algorithm to optimize the selection and assignment of medical imaging exams to the at least one peer reviewer using the received peer review data.

One embodiment provides a computer-implemented method for providing radiology peer review feedback and learning. The method includes: receiving a set of medical imaging exams with reading physician data, and at least one peer review score; training a machine learning algorithm to predict a review score from the medical imaging exams and reading physician data; and using the trained machine learning algorithm to represent the medical imaging exams and the reading physician data as feature vectors. The method further includes storing a history of feature vectors for the reviewed medical imaging exams for a reading physician; receiving newly-reviewed medical imaging exam data for the reading physician and representing a feature vector thereof; finding similar medical imaging exams in the history of the reviewed medical imaging exams by comparing the feature vector of the newly-reviewed medical imaging exam data with the feature vectors for the reviewed medical imaging exams for the reading physician, and providing common review feedback from the similar medical imaging exams to the reading physician.

Another embodiment provides a computer system for providing radiology peer review feedback and learning, using artificial intelligence. The computer system includes an electronic processor and one or more computer-readable memories. The electronic processor, through execution of instructions stored in the one or more computer-readable memories, is configured to: receive a set of medical imaging exams with reading physician data, and at least one peer review score; train a machine learning algorithm to predict a review score from the medical imaging exams and the reading physician data; use the trained machine learning algorithm to represent the medical imaging exams and the reading physician data as feature vectors; store a history of feature vectors for the medical imaging exams for a reading physician; receive newly-reviewed medical imaging exam data for the reading physician and represent a feature vector thereof; find similar medical imaging exams in the reading physician's history of the medical imaging exams by comparing the feature vector of the newly-reviewed medical imaging exam data with the feature vectors for the medical imaging exams for the reading physician; and provide common review feedback from the similar medical imaging exams to the reading physician.

Another embodiment is directed to a computer program product, the computer program product comprising a non-transitory computer readable storage medium having program code. The program code is executable as a set of instructions by an electronic processor to: receive a set of completed medical imaging exams with reading physician data, and at least one peer review score; train a machine learning algorithm to predict the review score from the medical imaging exam and reading physician data; use the trained machine learning algorithm to represent the medical imaging exams and the reading physician data as feature vectors; store a history of feature vectors for the reviewed medical imaging exams for a reading physician; receive newly-reviewed medical imaging exam data for the reading physician and represent a feature vector thereof; find similar medical imaging exams in the history of the reviewed medical imaging exams by comparing the feature vector of the newly-reviewed medical imaging exam data with the feature vectors for the reviewed medical imaging exams for the reading physician; and provide common review feedback from the similar medical imaging exams to the reading physician.

Other aspects of the invention will become apparent by consideration of the detailed description and accompanying drawings.

### BRIEF DESCRIPTION OF THE DRAWINGS

FIG. 1 illustrates a peer review system for performing peer review of medical imaging exams according to one embodiment.

FIG. 2 is a flowchart of a method performed by the system of FIG. 1 for maximizing peer review quality and minimizing excess time on peer reviews according to one embodiment.

FIG. 3 is a flowchart of a method performed by the system of FIG. 1 for providing radiology peer review feedback to a reading physician according to one embodiment.

FIG. 4 is a flowchart illustrating operation of another embodiment of the peer review system of FIG. 1.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and may include electrical connections or couplings, whether direct or indirect. Also, electronic communications and notifications may be performed using any known means including direct connections, wireless connections, etc.

A plurality of hardware and software based devices, as well as a plurality of different structural components may be utilized to implement the invention. In addition, embodiments of the invention may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects of the invention may be implemented in software (e.g., stored on non-transitory computer-readable medium) executable by one or more processors. As such, it should be noted that a plurality of hardware and software based devices, as well as a plurality of different structural components, may be utilized to implement the invention. For example, “mobile device,” “computer device,” and “server” as described in the specification may include one or more electronic processors, one or more memory modules including non-transitory computer-readable medium, one or more input/output interfaces, and various connections (e.g., a system bus) connecting the components.

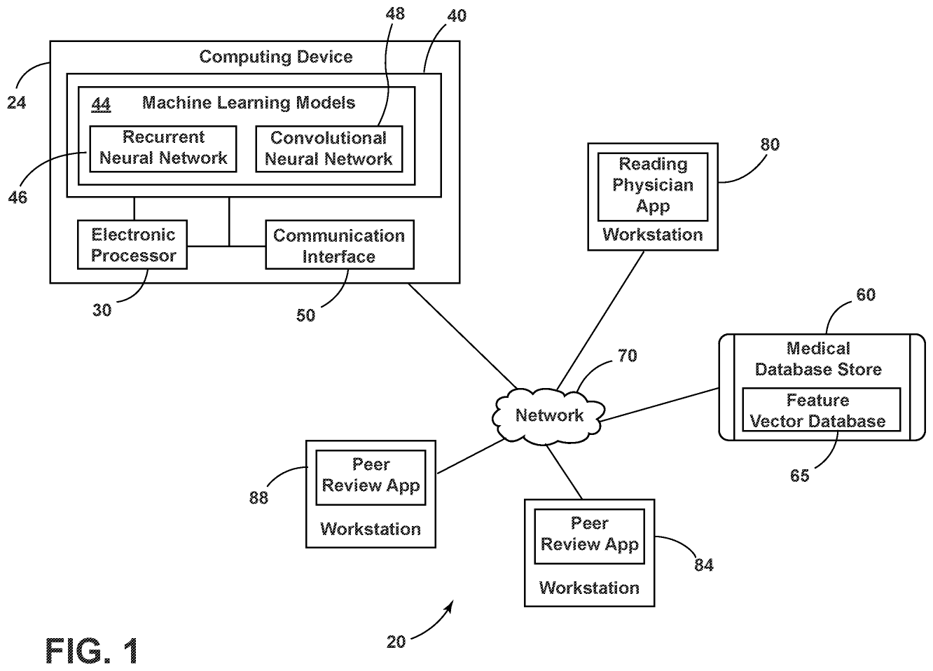

FIG. 1 illustrates a computer-implemented peer review system 20 for performing peer review of medical imaging exams read by reading physicians. In one embodiment, the peer review system 20 executes a picture archiving and communication system (PACS) computer program product for receiving medical imaging exams for study and analysis. A PACS is a high-speed computer network system for the storage, recovery, and display of radiological images.

The peer review system 20 shown in FIG. 1 includes a computer device 24 including a plurality of electrical and electronic components that provide power, operational control, and protection of the components within the computer device. In one embodiment, the computer device 24 is a server or other computer system (e.g., a PACS). For example, as illustrated in FIG. 1, the computer device 24 may include an electronic processor 30 (e.g., a microprocessor, application-specific integrated circuit (ASIC), or another suitable electronic device), a memory 40 (e.g., a non-transitory, computer-readable storage medium) that stores machine learning models 44, including, for example, a recurrent neural network 46 and a convolutional neural network 48. Other machine learning models are also contemplated. The computer device 24 also includes a communication interface 50. The electronic processor 30, the memory 40, and the communication interface 50 communicate over one or more connections or buses. The computer device 24 illustrated in FIG. 1 represents one example of a server and embodiments described herein may include a server with additional, fewer, or different components than the computer device 24 illustrated in FIG. 1. Also, in some embodiments, the computer device 24 performs functionality in addition to the functionality described herein. Similarly, the functionality performed by the computer device 24 (i.e., through execution of instructions by the electronic processor 30) may be distributed among multiple computer devices. Accordingly, functionality described herein as being performed by the electronic processor 30 may be performed by one or more electronic processors included in a server, external to the server, or a combination of computer devices.

The memory 40 may include read-only memory (“ROM”), random access memory (“RAM”) (e.g., dynamic RAM (“DRAM”), synchronous DRAM (“SDRAM”), and the like), electrically erasable programmable read-only memory (“EEPROM”), flash memory, a hard disk, a secure digital (“SD”) card, other suitable memory devices, or a combination thereof. One or more computer-readable memories are contemplated. The electronic processor 30 executes computer-readable instructions (“software”) stored in the memory 40. The software may include firmware, one or more applications, program data, filters, rules, one or more program modules, and other executable instructions for performing, among other things, the methods and functionality described herein. For example, as illustrated in FIG. 1, in some embodiments, the memory 40 also stores the machine learning models 44. As described in more detail below, the machine learning models 44 can include a recurrent neural network (RNN) 46 for processing sequences of data and providing predictions and/or a convolutional neural network (CNN) 48 for processing or analyzing raw image data in one embodiment. The functionality described herein as being performed by the neural networks may be distributed among multiple modules or applications (executed by the computer device 24 or multiple servers or computer devices).

The communication interface 50 allows the electronic processor 30 to communicate with devices external to the computer device 24. For example, as illustrated in FIG. 1, the computer device 24 may interact or communicate with a medical database store 60 through a communication network 70, wherein the medical database store 60 may include a feature vector database 65. In particular, the communication interface 50 may include a port for receiving a wired connection to an external device (e.g., a universal serial bus (“USB”) cable and the like), a transceiver for establishing a wireless connection to an external device over the communication network 70. The communication network 70 includes one or more communication networks (e.g., the Internet, a local area network (“LAN”), a wide area network (“WAN”), and the like), or a combination thereof.

In some embodiments, the computer device 24 acts as a gateway to the medical database store 60, a reading physician work station 80, and peer review workstations 84, 88. For example, in some embodiments, the computer device 24 includes a picture archiving and communication system (“PACS”) computer program having program code. The electronic processor 30 executes the program code to communicate with the medical database store 60 that includes medical imaging exams stored therein.

As illustrated in FIG. 1, computer device 24 also communicates with the reading physician workstation 80 (e.g., a personal computer device, such as, but not limited to, a laptop computer, a desktop computer, a terminal, a tablet computer, smart phone, a smart watch or other wearable, a smart television, and the like). The reading physician workstation 80 may communicate with the computer device 24 via the communication network 70. The reading physician workstation 80 may communicate with the computer device 24 to access one or more images stored in the medical database store 60. For example, a reading physician may use a browser application executed by the computer device 24 to access a web page provided by the computer device for accessing (viewing) one or more images. In other embodiments, the reading physician may use a dedicated application executed by the reading physician workstation 80 (a viewer application) to retrieve images from the medical database store 60.

Likewise, as illustrated in FIG. 1, the computer device 24 also communicates with the peer review workstations 84, 88 (e.g., personal computing devices, such as, but not limited to, a laptop computer, a desktop computer, a terminal, a tablet computer, smart phone, a smart watch or other wearable, a smart television, and the like). The peer review workstations 84, 88 may communicate with the computer device 24 via the communication network 70. The peer review workstations 84, 88 may communicate with the computer device 24 to access medical imaging exams including reading physician data stored in the medical database store 60. For example, a peer reviewer may use a browser application executed by the peer review workstation 84, 88 to access a web page provided by the computer device 24 for accessing (viewing) one or more medical imaging exams and reading physician data. In other embodiments, the peer reviewer may include a dedicated application executed by the reading physician workstation 80 (a viewer application) to retrieve medical imaging exams and reading physician data from the medical database store 60.

As illustrated in FIG. 1, in some embodiments the reading physician workstation 80 and the peer review workstations 84, 88, each include similar components as the computer device 24, such as an electronic processor 30, a memory 40, and a communication interface 50 for communicating with external devices, such as via the communication network 70. The reading physician workstation 80 and the peer review workstations 84, 88, each include at least one output device, such as one or more display devices, one or more speakers, and the like configured to provide output to a user, and at least one input device, such as a microphone, a keyboard, a cursor-control, device, a touchscreen, or the like configured to receive an input from a reading physician or a peer reviewer.

### REVIEW PROCESS

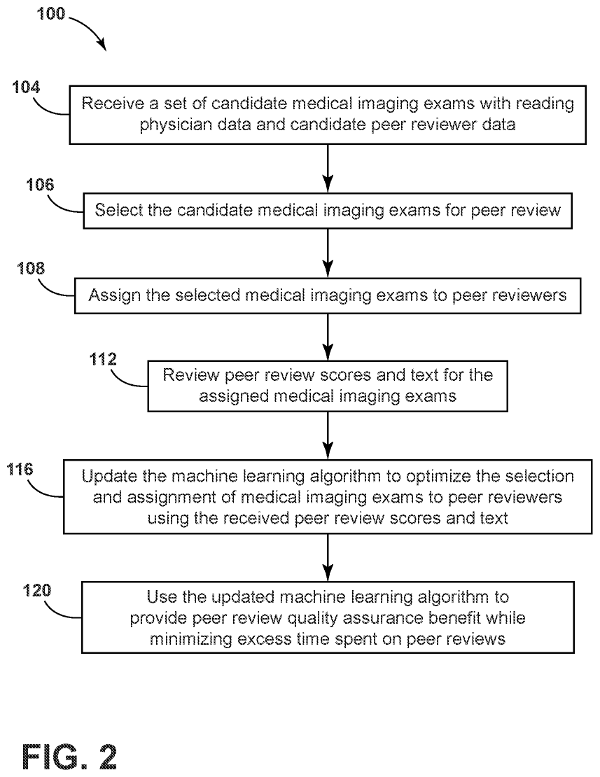

In one embodiment, the computer device 24 generates a random peer review worklist from a database table of available peer reviewers (e.g., on request). In other embodiments, however, the computer device 24 is configured to use one or more machine learning algorithms to select a medical imaging exam for review and, optionally, one or more peer reviewers for reviewing the selected medical imaging exam. This selection can be performed during current exam reading workflow. For example, in some embodiments, a review can be recommended for a current medical imaging exam when a relevant prior exam (for the same patient) was viewed for the current exam. Recommended reviews can be requested based on configurable triggers or timing, such as, for example, during prior viewing, when a current image study is marked read, or the like. The medical imaging exam selections selected via AI can be based on exam specifics (e.g., medical images, report text using natural language processing (NLP), and reader characteristics), reviewer characteristics, or combinations thereof. Further details regarding exam selection and assignment to one or more reviewers are provided below with respect to FIG. 2.

After a medical imaging exam is selected for review and a reviewer is assigned, embodiments described herein can provide a user interface that allows a reviewer to efficiently and effectively provide feedback on the medical imaging exam. For example, when completing a medical imaging exam (i.e., generating an exam report), a reading physician at the reading physician workstation 80 studies a medical imaging exam and enters appropriate reading physician data as a result of the review into an automatically populated interface. Likewise, subsequent reviewers at peer review workstations 84, 88 receive completed medical imaging exams with reading physician data in an automatically populated interface. The reading physician data may include, for example, study date and exam descriptor details in an automatically populated interface. A reviewer that agrees with the original report has a single click option to provide a score (e.g., a score within a predetermined scoring scheme, such as, for example, a RADPEER score of 1). Thus, ease of use is provided for an assigned peer reviewer using an automatically populated interface by a single click to agree with the reading physician data provided by the reading physician. Further, the PACS system includes an option to provide positive feedback or comments. In instances where a peer reviewer disagrees with a reading physician, a different score is provided (e.g., a RADPEER score of 2 or 3), and comments from the peer reviewer are typically required. Thus, the peer reviewer provides text when the peer reviewer does not agree with the reading physician data provided by the reading physician for a completed medical imaging exam.

Collected peer review information can be viewable by a quality assurance lead, the original reading physician, or both. Various levels of reviewer anonymity can be used as a configurable parameter of the system. In some embodiments, the reviewee/reading physician can view review information and can agree with the review or disagree or contest the review (e.g., entering a reason as text). In some embodiments, a quality assurance lead can adjudicate disagreements and provide a final say on whether the reviewer or the reviewee was correct. The review information, the reviewer's agreement, disagreement, and any explanation text can be used to train (e.g., further update) the machine learning algorithms (as described in more detail below). As also described in more detail below, the review information (and reviewee feedback) can also be used to learn trends for readers, reviewers, or both, which allows the AI system to confidentially inform readers of their common errors or situations that lead to errors. Collected review information can also be summarized or aggregated and uploaded to one or more accrediting bodies, such as RADPEER.

FIG. 2 is a flowchart 100 of a first method performed by the system 20 shown in FIG. 1. At a first step 104, the computer device 24 receives a set of candidate medical imaging exams with reading physician data from the medical database store 60, such as a PACS or other memory device. At step 106, the computer device 24 selects at least a subset of the candidate medical imaging exams for peer review. The selection is based on various criteria based on one or more machine learning algorithms. In some embodiments, the machine learning algorithm can be configured to predict a review score for each candidate imaging exam (e.g., using training information including completed reviews of other imaging exams) and uses the predict scores to select at least a subset of the candidate medical image exams for review. As described below, some embodiments can use online machine learning. In these embodiments, selecting medical image exams can initially be done randomly or based on predefined rules. Thereafter, the updates by the online machine learning algorithm adjust a selection threshold.

In one embodiment, the machine learning algorithm recommends exams with high scores for peer review based on a threshold value. In one embodiment, the machine learning algorithm performs online calibration of the threshold value to maintain a desired percentage of peer reviews for the medical imaging exams. In one embodiment, a site administrator can also configure the desired percentage of peer reviews for the candidate medical imaging exams to be reviewed.

At step 108, the computer device 24 analyzes the selected medical imaging exams and the reading physician data and utilizes the one or more machine learning algorithms to, assign each of the selected medical imaging exams to at least one peer reviewer. Peer reviewers can be selected based on their ability to analyze certain types of medical imaging exams in one embodiment.

At step 112, the computer device 24 receives peer review data from assigned reviewers. In one embodiment, the peer review data includes scores and/or text for the assigned medical imaging exams delivered through an automatically populated interface from the peer review workstations 84, 88 as described above.

At step 116, the computer device 24 executes an online update for updating the one or more machine learning algorithms used to select and assign medical imaging exams to the peer reviewers. The machine learning algorithm is configured to analyze various categories of candidate medical imaging exams and to determine the types of medical imaging exams that are more prone to errors. The machine learning algorithm determines what types of mistakes each reading physician makes and in what situations or at what times. The machine learning algorithm determines which peer reviewers are good at determining what types of mistakes by reading physicians on what types of medical imaging exams. Therefore, the assigning of the medical imaging exams to the selected peer reviewers includes assigning the medical imaging exam to at least one peer reviewer most competent for that type of medical imaging exam.

It should be understood that medical imaging exams with a higher score are more likely prone to errors, and such exams may be reviewed by more peer reviewers than a basic medical imaging exam. Accordingly, the machine learning algorithms described above can be configured to determine which of the peer reviewers are most competent at discovering errors and on which types of medical imaging exams. The machine learning algorithm also utilizes ground truth review of exam scores from completed reviews to learn to predict peer review scores.

As illustrated in FIG. 2, step 120 of the method 100 includes using the trained machine learning algorithm executed by the computer device 24 to simultaneously provide peer review quality assurance benefit while minimizing excess time on peer reviews. In other words, as the machine learning algorithms are trained or learn (e.g., updated using online learning), the one or more updated machine learning algorithms can select medical imaging exams for peer review that are more likely to have potential errors and/or are more complicated than other medical imaging exams. Similarly, once properly trained or updated, the one or more updated machine learning algorithms can assign selected medical imaging exams to the peer reviewers most capable of conducting a peer review depending on the type of medical imaging exam, the qualifications of the peer reviewers, and past review scores of the peer reviewers. Accordingly, using the trained machine learning algorithms, medical imaging exams that are considered basic or straight forward and, thus, unlikely to have issues are peer reviewed by fewer peer reviewers or not reviewed at all in some embodiments.

In some embodiments, the functionality illustrated in FIG. 2 and described above is provided as a computer program product that includes a non-transitory computer readable storage medium having program code. The program code is executable as a set of instructions by an electronic processor 30 of a computer device 24 to perform the steps shown in FIG. 2.

### PEER REVIEW FEEDBACK

As noted above, in addition to creating efficiencies in exam selection, reviewer assignment, and review collection or input, embodiments described herein can alternatively or in addition create efficiencies and improvements in using collected reviews to provide useful feedback to a reading physician and providing such feedback in a way that incentives reviewers to provide accurate and truthful feedback.

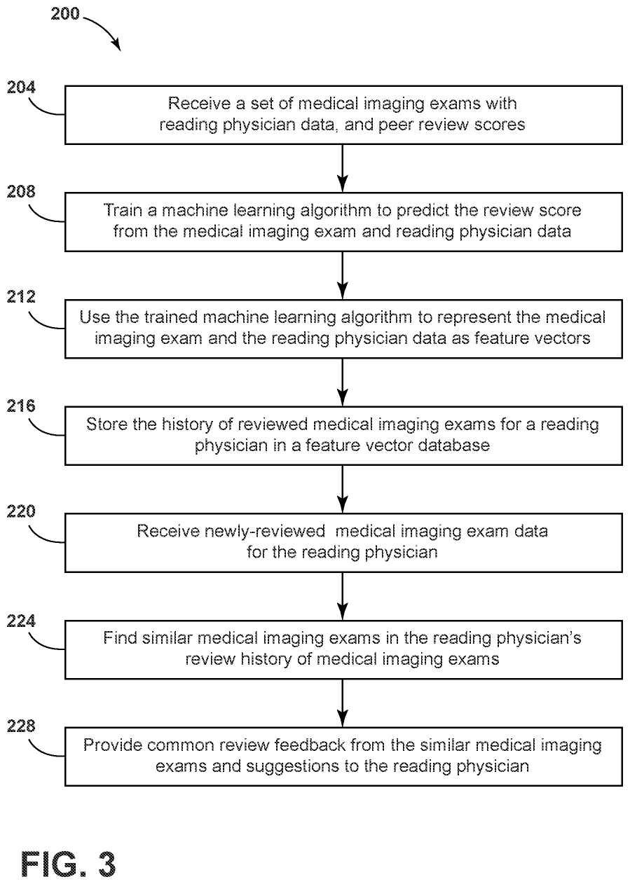

For example, FIG. 3 is a flowchart 200 of a peer review feedback method performed by the system 20 shown in FIG. 1. At a first step 204, the computer device 24 gathers or receives a set of completed medical imaging exams with reading physician data, at least one peer review score, and in some instances, peer review explanation text from the medical database store 60, such as a PACS or other memory device. The computer device 24 advances to step 208.

At step 208, the computer device 24 trains a machine learning algorithm to predict a review score from or based on the medical imaging exam and the reading physician data. The peer review scores are provided to assist in the training of the machine learning algorithm (e.g., supervised learning). In particular, the computer device 24 can train a classifier to convert input data (exam, reading physician, and review data (scores)) into a feature presentation and predict a review score for each reader. After the training is complete, the computer device 24 advances to step 212.

At step 212, the computer devices uses the trained machine learning algorithm to represent the medical imaging exam and the reading physician data as feature vectors. The peer review scores can also be stored as part of a feature vector. In one embodiment, a feature vector is an n-dimensional vector of numerical features that describe some object in pattern recognition in machine learning. Thus, a feature vector is a list of numerical values/calculated values. Feature vectors are especially useful for image processing analysis. The computer device 24 advances to step 216.

At step 216, the computer device 24 stores the history of reviewed medical imaging exams for a reading physician as feature vectors in a feature vector database 65. FIG. 1 shows the feature vector database 65 as part of the medical database store 60. However, a separate memory may also provide the feature vector database 65. Steps 204, 208, 212, 216 are provided for a plurality of completed medical imaging exams of the reading physician. Thus, a history of feature vectors corresponding to various reviewed medical imaging exams for the reading physician are stored in the feature vector database 65. The computer device 24 advances to step 220.

At step 220, the computer device 24 is configured to receive newly-reviewed medical imaging exam data for a medical imaging exam for the reading physician. In one embodiment, the machine learning algorithm executed by the computer device 24 represents the newly-reviewed medical imaging exam data as a feature vector. The computer device 24 advances to step 224.

At step 224, the computer device 24 finds similar medical imaging exams in the reading physician's history of medical imaging exams. In one instance, the similar medical imaging exams are determined by comparing the feature vector of the newly-reviewed medical imaging exam data with the feature vectors for similar medical imaging exams for the reading physician that are stored in the feature vector database 65. The computer device 24 advances to step 228.

At step 228, the computer device 24 provides common review feedback from the similar medical imaging exams and suggestions to the reading physician. In one embodiment, the common review feedback from the similar medical imaging exams and suggestions are provided to a quality lead person. In another embodiment, the machine learning algorithm analyzes other environment features including time of day and exam details for other medical exams to provide trends to the reading physician. One possible trend is a time of day wherein the reading physician is more likely to make an error. In one embodiment, the suggestions include common misses by the reading physician or other physicians for the type of medical exam being reviewed by the reading physician. In one embodiment, we graphically show where missed findings are commonly located on images/anatomy and show common review feedback as text.

In one embodiment, the machine learning algorithm is an online reinforcement learning algorithm. Another embodiment provides anonymity for peer reviewers.

As noted above, the data input to the machine learning algorithm for selecting an imaging exam for review can include exam details, reader details, reviewer details. This information can be stored in the system (e.g., a PACS database), various logs maintained by the system 20 or other systems, completed reports (e.g., analyzed using natural language processor). For example, characteristics of a reader and a reviewer can include a user's specialty, modalities, shift schedules, etc. Details of the imaging exam can include a body part, impressions, findings, annotations, measurements, anatomy segmentation, modality, procedure, priors, number of slices, computer-aided diagnosis (CAD) results, raw image data, or the like. Other input data can include environment features, such as, for example, time of day, exam details for other exams the reading physician read before and after reading the exam under consideration for review, whether the reading physician also read prior exams when they exist, etc. In some embodiments, different machine learning algorithms can be used for combinations of the above input data. For example, in some embodiments, one machine learning algorithm can be configured to process raw image data of a candidate report (e.g., the CNN 48) and a separate machine learning algorithm (e.g., the RNN 46) can be configured to process other input data, such as, for example, prior exam information. The output of each machine learning algorithm can be combined or processed in various ways. For example, in some embodiments both machine learning algorithms generated a predicted review score and the scores can be combined, such as by averaging, to determine a final predicted review score for an imaging exam.

The machine learning algorithm(s) used by the system 20 can be trained using training data including the input data generally described above. Ground truth review scores can also be automatically determined calculated for the training data. These scores can include actual review scores or, in other embodiments, can be based on multiple factors. These factors can include a RADPEER score or other peer review scoring system (e.g., wherein a 1 represents a low score and a 3.b represents a highest score), a quality of review (e.g., based on a time spent or images viewed during a review of an imaging exam in the training data, a confirmation of diagnosis by follow-up or biopsy (if available), a complexity or difficulty score (e.g., a relative value unit (RVU)), or a combination thereof.

Once trained the machine learning algorithms can be used to select a subset of candidate completed imaging exam for review and can assign a selected exam to a particular reviewer. As noted above, in some embodiments, the machine learning algorithms used by the system 24 can learn specialties, preferences.

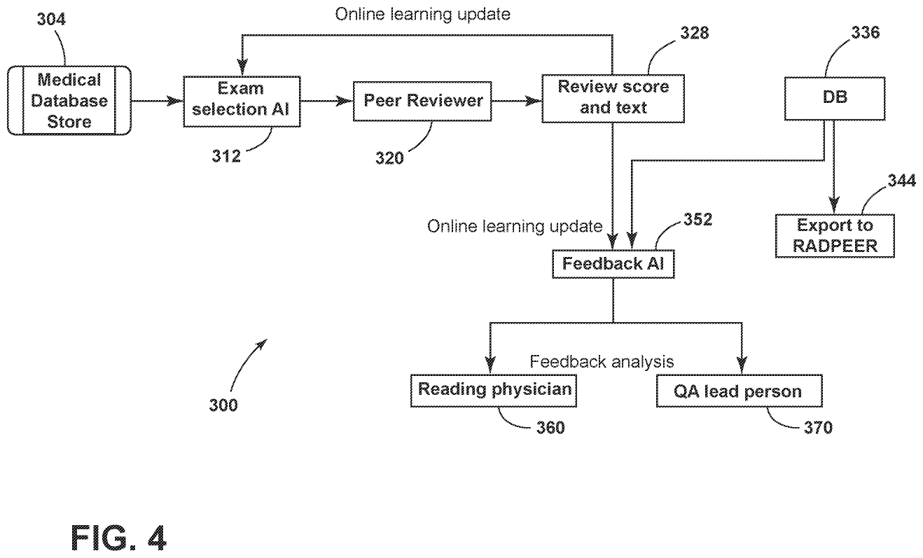

FIG. 4 shows a flowchart 300 illustrating another embodiment of the peer review system with similarities to the online machine learning arrangement of FIG. 2. A medical database store 304 provides a set of candidate medical imaging exams with reading physician data to exam selection (artificial intelligence) 312. An online machine learning algorithm is applied to determine which exams to for review at exam selection step 312. The online machine learning algorithm selects the medical imaging exams for review randomly or based on predefined rules before any online training/updating has happened. Over time, the medical imaging exams with high scores will be selected for peer review based on a threshold value. Medical imaging exams with lower scores are not evaluated further, as there is less likelihood of error and thus excess time spent on peer reviews having no likely error is minimized.

Assuming a candidate medical imaging exam is selected, the flowchart 300 advances to peer reviewer step 320, wherein at least one peer reviewer is assigned to review the selected medical imaging exam. Before any online updating or training has occurred, the assignment of a peer reviewer may be done randomly or based on predefined rules. The peer reviewer provides a review score and, in some instances, explanation text when appropriate as shown at step 328. The review score and occasional explanation text is fed back to the exam selection step 312 to provide an online learning update for training the machine learning algorithm that operates to optimize the selection and assignment of medical imaging exams. In one embodiment, ground truth scores are calculated based on the review scores themselves or including other factors like review time spent by the reviewer. The input data, and ground truth score are used to update the online machine learning algorithm. If the online machine learning algorithm is a neural network, the update will be provided to the network weights using gradient descent. In another embodiment, the updating of the machine learning algorithm is an online machine learning update. Other arrangements are contemplated.

Further, the review score and text at step 328 can be provided to a database 336 or other memory for export to a RADPEER database as shown at step 344 to meet requirements from the American College of Radiology (ACR). Further, the review score and text for the selected medical imaging exam at step 328 is also provided, at feedback step 352 to the reading physician 360 and/or to a quality assurance (QA) lead person 370. The feedback includes common review feedback from similar medical imaging exams of the reading physician, and in some embodiments includes providing suggestions. Thus, the machine learning algorithm is operating to provide feedback.

In some embodiments, the steps described above, are predefined for one or multiple reading physicians. Alternatively or in addition, the steps may be initially created or modified using machine learning. Machine learning generally refers to the ability of a computer program to learn without being explicitly programmed. In some embodiments, a computer program (e.g., a learning engine) is configured to construct a model (e.g., one or more algorithms) based on example inputs. Supervised learning involves presenting a computer program with example inputs and their desired (e.g., actual) outputs. The computer program is configured to learn a general rule (e.g., a model) that maps the inputs to the outputs. The computer program may be configured to perform deep machine learning using various types of methods and mechanisms. For example, the computer program may perform deep machine learning using decision tree learning, association rule learning, artificial neural networks, inductive logic programming, support vector machines, clustering, Bayesian networks, reinforcement learning, representation learning, similarity and metric learning, sparse dictionary learning, and genetic algorithms. Using all of these approaches, a computer program may ingest, parse, and understand data and progressively refine models for data analytics.

Thus, embodiments described herein provide, among other things, methods and systems for improving radiology peer review using artificial intelligence. Machine learning techniques may be used to establish or modify such rules, which further improve the efficiency and effectiveness of the systems and methods. Various features and advantages of the invention are set forth in the following claims.

What is claimed is:

1. A computer-implemented method for optimizing radiology peer review exam selection using artificial intelligence, the method comprising:
receiving a set of candidate medical imaging exams with reading physician data;
selecting the candidate medical imaging exams for peer review;
assigning the selected medical imaging exams to at least one peer reviewer;
receiving peer review data from the at least one peer reviewer assigned to the selected medical imaging exams, the peer review data including at least one score for the medical imaging exams; and
updating a machine learning algorithm to optimize the selection and assignment of medical imaging exams to the at least one peer reviewer using the received peer review data.

2. The method of claim 1, wherein the selecting of the candidate medical imaging exams for peer review includes operating the machine learning algorithm to select the medical imaging exams with high scores for peer review based on a threshold value.

3. The method of claim 2, wherein the selecting of the candidate medical imaging exams for peer review includes operating the machine learning algorithm to perform online calibration of the threshold value to maintain a desired percentage of peer reviews for the medical imaging exams.

4. The method of claim 3, wherein the desired percentage of peer reviews for the medical imaging exams is provided by an input received from a site administrator.

5. The method of claim 1, wherein the assigning of the selected medical imaging exams to the at least one peer reviewer includes using the updated machine learning algorithm to determine which of the peer reviewers are most competent at discovering errors on which types of the medical imaging exams, and wherein the updating of the machine learning algorithm is an online machine learning update.

6. The method of claim 5, wherein the using of the machine learning algorithm to optimize the assigning of the medical imaging exams to the at least one peer reviewer includes assigning the medical imaging exam to the at least one peer reviewer most competent for that type of the medical imaging exam.

7. The method of claim 1, including using the machine learning algorithm to determine which types of the medical imaging exams are more prone to errors, and wherein the machine learning algorithm utilizes ground truth review of exam scores from completed reviews of the medical imaging exams to learn to predict peer review scores.

8. A computer system for optimizing radiology peer review exam selection using artificial intelligence, the computer system comprising:
an electronic processor; and
one or more computer-readable memories,
wherein the electronic processor, through execution of instructions stored in the one or more computer-readable memories, is configured to:
receive a set of candidate medical imaging exams with reading physician data;
select the candidate medical imaging exams for peer review;
assign the selected medical imaging exams to at least one peer reviewer;
receive peer review data from the at least one peer reviewer assigned to the selected medical imaging exams, the peer review data including at least one score for the medical imaging exams; and
update a machine learning algorithm to optimize the selection and assignment of medical imaging exams to the at least one peer reviewer using the received peer review score.

9. The system of claim 8, wherein the selecting of the candidate medical imaging exams for peer review includes operating the machine learning algorithm to select the medical imaging exams with high scores for peer review based on a threshold value.

10. The system of claim 9, wherein the selecting of the candidate medical imaging exams for peer review includes operating the machine learning algorithm to perform online calibration of the threshold value to maintain a desired percentage of peer reviews for the medical imaging exams.

11. The system of claim 10, wherein the desired percentage of peer reviews for the medical imaging exams is provided by an input received from a site administrator.

12. The system of claim 8, wherein the assigning of the selected medical imaging exams to the at least one peer reviewer includes using the machine learning algorithm as updated to determine which of the peer reviewers are most competent at discovering errors on which types of the medical imaging exams.

13. The system of claim 12, wherein the using of the machine learning algorithm to optimize the assigning of the selected medical imaging exams to the at least one peer reviewer includes assigning the medical imaging exams to the at least one peer reviewer most competent for that type of the medical imaging exams.

14. The system of claim 8, including using the machine learning algorithm to determine which types of the medical imaging exams are more prone to errors, and wherein the machine learning algorithm utilizes ground truth review of exam scores from completed peer reviews of the medical imaging exams to learn to predict peer review scores, and wherein the reading physician data includes explanation text and the peer review data includes explanation text.

15. A computer program product, the computer program product comprising a non-transitory computer readable storage medium having program code, the program code executable by an electronic processor to:
receive a set of candidate medical imaging exams with reading physician data;
select the candidate medical imaging exams for peer review;
assign the selected medical imaging exams to at least one peer reviewer;
receive peer review data from the at least one peer reviewer assigned to the selected medical imaging exams, the peer review data including at least one peer review score for the assigned medical imaging exams; and
update a machine learning algorithm to optimize the selection and assignment of the medical imaging exams to the at least one peer reviewer using the received peer review data.

16. The computer program product of claim 15, wherein the selecting of the candidate medical imaging exams for peer review includes operating the machine learning algorithm to select the medical imaging exams with high scores for peer review based on a threshold value.

17. The computer program product of claim 16, wherein the selecting of the candidate medical imaging exams for peer review includes operating the machine learning algorithm to perform online calibration of the threshold value to maintain a desired percentage of peer reviews for the medical imaging exams, and wherein the updating of the machine learning algorithm is an online machine learning update.

18. The computer program product of claim 15, wherein assigning of the selected medical imaging exams to the at least one peer reviewer includes using the machine learning algorithm to determine which of the peer reviewers are most competent at discovering errors on which types of the medical imaging exams.

19. The computer program product of claim 18, wherein the operating of the machine learning algorithm to optimize the assigning of the medical imaging exams to the at least one peer reviewer includes assigning the medical imaging exam to the at least one peer reviewer most competent for that type of medical imaging exam.

20. The computer program product of claim 15, including using the machine learning algorithm to determine which types of the medical imaging exams are more prone to errors, and wherein the machine learning algorithm utilizes ground truth review of exam scores from completed peer reviews of the medical imaging exams to learn to predict peer review scores.
